(19) United States
(12) Patent Application Publication
(10) Pub. No.: US 2011/0224276 A1
(43) Pub. Date: Sep. 15, 2011

(54) OPTICAL ISOMER OF PHENYLPROPIONIC ACID AND ITS MEDICINAL USE

(76) Inventors: Tao Wang, Hefei City (CN); Yan Geng, Hefei City (CN); Zonggui Wang, Hefei City (CN); Hongzhang Sun, Hefei City (CN)

(21) Appl. No.: 13/130,603

(22) PCT Filed: Nov. 20, 2009
(86) PCT No.: PCT/CN09/75058

§ 371 Date: May 23, 2011

(30) Foreign Application Priority Data
Nov. 21, 2008 (CN) 200810244303.2

(51) Int. Cl. A61K 31/38; C07D 337/14; A61P 29/00; A61P 19/02

(52) U.S. Cl. 514/431; 549/12

(57) ABSTRACT

Optical isomers of phenylpropionic acid drugs are a mixture having R-type optical isomer and S-type optical isomer at a ratio of 10:1-1:10 by weight, wherein the ratio of 1:1 is excluded. A use of the optical isomers includes that the R-type optical isomer is used for manufacturing anti-inflammatory and analgesic drugs and the mixture having the R-type optical isomer and the S-type optical isomer at a ratio of 10:1-1:10 by weight, wherein the ratio of 1:1 is excluded, is used for manufacturing anti-inflammatory and analgesic drugs. The therapeutic indexes of anti-inflammatory and analgesic drugs of the present optical isomers are all higher than those of the S-type optical isomers and the racemate.

### TECHNICAL FIELD

The present invention relates to a drug compound of single optical isomer and medical uses thereof, and in particular, relates to R-type single optical isomer of 10,11-dihydro-alpha-methyl-10-oxo-dibenzo[b,f]tiazem-2-acetic acid and medical uses thereof, as well as the medical uses of the mixture having R-type optical isomer and S-type optical isomer at different ratios.

### BACKGROUND ART

10,11-dihydro-alpha-methyl-10-oxo-dibenzo[b,f]tiazem-2-acetic acid is developed by Japanese Chemiphar, which is a powerful non-steroidal anti-inflammatory, analgesic, febrifuge firstly sold in Japan in 1993 under the trade name of Soleton. This drug selectivity acts on inflammation sites, and will not affect other organs such as stomach and kidney. Further, this drug has advantages such as high efficiency, small side effect on stomach, etc., compared with congener drugs such as Naproxen and Ibuprofen, and this drug has an abirritation of 3-28 times more than Indomethacin, Diclofenac Sodium, Ketoprofen, mefenamic acid, etc. Therefore, this drug is wildly appreciated in the clinic. Its mechanism is to inhibit the activity of epoxidase, reduce the syntheses of prostaglandin, and block inflammatory media, further, this drug has membrane stabilizing action such as leucocyte migration inhibition, lysosomal enzyme liberation inhibition, etc.

10,11-dihydro-alpha-methyl-10-oxo-dibenzo[b,f]tiazem-2-acetic acid is a non-steroidal anti-inflammatory drug of phenylpropionic acid, which has one chiral center in the structure, and 10,11-dihydro-alpha-methyl-10-oxo-dibenzo[b,f]tiazem-2-acetic acid sold in Japan is its racemic mixture, i.e., a mixture of S-type one and R-type one at equal proportion.

The patent application (CN application No. 200810020558.0) discloses a preparation method of 10,11-dihydro-alpha-methyl-10-oxo-dibenzo[b,f]tiazem-2-acetic acid optical isomers, and R-type (formula I) and S-type (formula II) optical isomers are obtained from racemate by a process of chiral resolution. This application shows that the anti-inflammatory and analgesic activity of S-type isomer is far higher than that of R-type by a pharmacodynamic test. This application also discloses the medical uses of S-type isomer in anti-inflammation and analgesia.

By pharmacodynamic and toxicologic tests on R-type isomer, S-type isomer, and a mixture thereof with different proportions, the applicant surprisingly discovers that R-type isomer has a lower anti-inflammatory and analgesic activity than that of S-type isomer, but its toxicity is even lower than that of S-type isomer, and therapeutic index of R-type isomer in anti-inflammation and analgesia is far higher than S-type isomer and their racemate. Further studies show that therapeutic index of the mixture of R-type and S-type isomer with different proportions (excluding 1:1) also higher than S-type isomer and their racemate.

Based on results of pharmacodynamic and toxicologic tests, the applicant therefore obtained a inspiration of modifying 10,11-dihydro-alpha-methyl-10-oxo-dibenzo[b,f]tiazem-2-acetic acid racemate to R-type single optical isomer or a mixture of R-type isomer and S-type isomer with different proportions (i.e. excluding the mixture with equal ratio of isomers) so as to obtain better anti-inflammatory and analgesic effect in the clinic, meanwhile to reduce the adverse reaction.

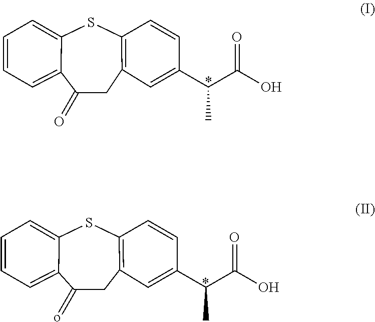

Pharmaceutical acceptable inorganic salts thereof are represented by following formula:

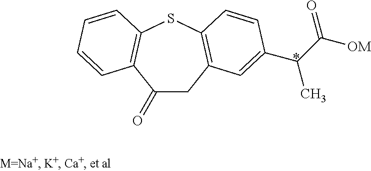

Wherein * represents R optical isomer, S optical isomer, R,S optical isomers with different proportions, and racemate.

The preparation principle can be represented by the following reaction formula:

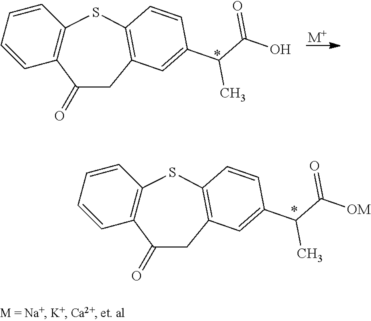

Pharmaceutical acceptable amino acid salts thereof are represented by following formula:

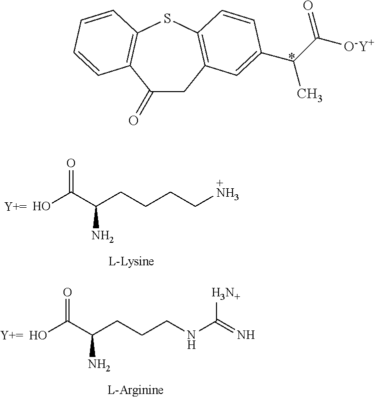

wherein * represents R optical isomer, S optical isomer, R, S optical isomers with different proportions, and racemate.

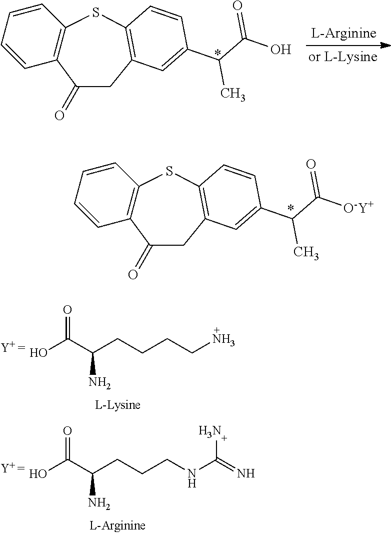

Contents of the Invention

The present invention intends to provide a drug in the same class but having more therapeutic index in anti-inflammation and analgesia than that of 10,11-dihydro-alpha-methyl-10-oxo-dibenzo[b,f]tiazem-2-acetic acid (hereinafter, referred to as phenylpropionic acid drugs, and the optical isomers thereof are referred to as R-type optical isomer of phenylpropionic acid drugs and S-type optical isomer of phenylpropionic acid drugs, respectively, or are simply called R-type optical isomer and S-type optical isomer, respectively) racemate, and the technical problem to be solved is to select its optical isomers or a mixture of optical isomers with an unequal ratio by means of pharmacodynamic and toxicologic tests.

The optical isomers of phenylpropionic acid drugs defined by the present invention means a mixture (hereinafter, called a mixture of isomers with different proportions) of R-type optical isomer or its pharmaceutical salts and S-type optical isomer or its pharmaceutical salts with a ratio of 10:1-1:10, wherein a mixture at a ratio of 1:1 is excluded.

The medical uses of optical isomers of phenylpropionic acid drugs defined by the present invention means the medical uses of single R-type optical isomer or its pharmaceutical salts, or the medical uses of a mixture of R-type optical isomer or its pharmaceutical acceptable salts and S-type optical isomer or its pharmaceutical acceptable salts, at a ratio of 10:1-1:10 by weight, wherein the mixture with a ratio of 1:1 is excluded. Said pharmaceutical acceptable salts include, but not limit to, sodium salts, potassium salts, calcium salts, amino acid salts, etc.

Based on the results of pharmacodynamic and toxicologic tests, the present invention selects out R-type single optical isomer or its pharmaceutical salts, as represented by the following chemical formula (I):

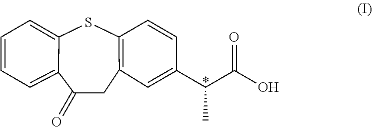

In the formula, chiral center (*) is R-type, and said pharmaceutical acceptable salts include, but not limit to, sodium salts, potassium salts, calcium salts, amino acid salts, etc.

The pharmaceutical acceptable inorganic salts are represented by following formula:

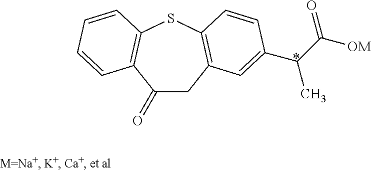

wherein * represents R optical isomer, S optical isomer, R,S optical isomers with different ratios, and racemate.

The preparation principle can be represented by the following reaction formula:

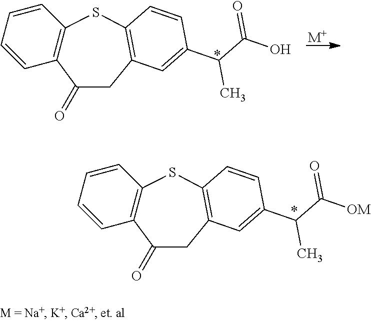

The pharmaceutical acceptable amino acid salts are represented by following formula:

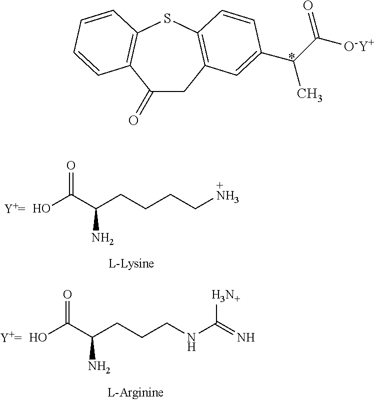

wherein * represents R optical isomer, S optical isomer, R,S optical isomers with different ratios, and racemate.

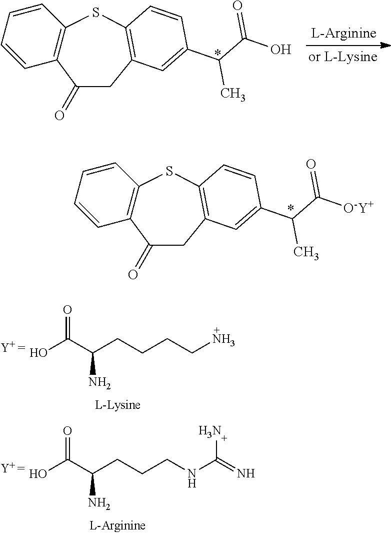

Epoxidase-1 (Cox-1), epoxidase-2 (Cox-2) and 5-lipoxidase (5-Lox) are important factors which mediate pain and inflammatory reaction. It is showed that existing racemate (RS) inhibits to the Cox-1, Cox-2 and 5-Lox at different degrees respectively, wherein the inhibiting effect to the Cox-2 is the most, and the racemate inhabits to the three enzymes at a larger extent than to the S-type optical isomer (S) and R-type optical isomer (R). As a whole, among other ratios (S:R) of samples, the samples with ratios of 6:1, 4:1, 1:2, 1:4, 1:6 and the like have more effects than RS (1:1).

By test of writhing in mice induced by acetic acid, 50% of maximum effective dosages of analgesic effect of tested drugs via intragastric administration (ED50, mg/kg) are determined, meanwhile the half-lethal doses of tested samples on mice via intragastric administration (LD₅₀, mg/kg) are determined, to further calculate therapeutic index (LD₅₀/ED50). The results show that R-type isomer has higher therapeutic index in anti-inflammation and analgesia than RS, and the therapeutic indexes of RS, R, S, and S:R=8:1, 6:1, 4:1, 2:1, 1:2, 1:4, 1:6, 1:8 are 35.5, 157.2, 31.2, 39.8, 43.2, 57.1, 50.4, 81.5, 105.6, 165.0, 158.0, respectively, wherein the therapeutic indexes of five samples including R, S:R=1:2, 1:4, 1:6, 1:8 all are twice or more than that of RS sample, and the sample with S:R=1:6 has the highest therapeutic index. Said anti-inflammatory and analgesic effects are mainly used to clinically treat chronic rheumatoid arthritis, arthritis deformans, low back pain, neck pain, cervico-omo-brachial syndrome, periarthritis humeroscapularis, as well as post-operative or post-trauma analgesia, dephlogisticate aspect.

Long-term toxicity tests of the rats subjected to four-week consecutive intragastric administration (ig) show that the main toxic target organs of large dose long-term administration of each of Zaltoprofen samples are gastrointestinal tracts, and toxic degree has a significant dose-dependent property. Nontoxic dosages of RS, R, S and R:S=6:1 in the rats subjected to four-week consecutive intragastric administration are 40, 240, 20 and 160 mg/kg, respectively. Based on this criterion, the safeties of R-Zaltoprofen and R:S=6:1 mixture are more than six times greater than, and more than four times greater than that of RS-Zaltoprofen, respectively.

Long-term toxicity tests of the rats subjected to 13-week consecutive intragastric administration and four-week convalescence show that the main toxic target organs of large dose long-term administration of each of Zaltoprofen samples are gastrointestinal tracts, toxic degree has a significant dose-dependent property, and the changes caused by most drugs can be recovered after drug withdrawal, i.e., reversible. With combination of each tested index, Nontoxic dosages of RS, R, S and R:S=6:1 in the rats subjected to 13-week consecutive intragastric administration are 25, 200, 12.5 and 100 mg/kg, respectively. Based on this criterion, the safeties of R-Zaltoprofen and R:S=6:1 mixture of 13-week consecutive intragastric administration of rats are more than six times greater than, and more than four times greater than that of RS-Zaltoprofen, respectively.

Ig pharmacokinetic studies of R-type optical isomer in rats show that ig pharmacokinetic behavior of R-type optical isomer is well, and corresponds to two-compartment model, and there are certain linear relationship among plasma drug concentration, AUC and dosage. After intragastric administration, the drugs are introduced into blood and distributed quickly, and the elimination of drug is relatively slow. No transformation between R-isomer and S-isomer is detected during the monitoring of blood drug level. Combination this result with the results of activity and toxicity evaluation shows that R-type isomer exerts its related function in vivo by itself other than the components after transformation, and has independent biological activity.

Based on the above experimental results, so called the medical uses of optical isomers of phenylpropionic acid drugs are: firstly, use of R-type optical isomer or its pharmaceutical acceptable salts for manufacturing anti-inflammatory and analgesic drugs, including the use for manufacturing anti-inflammatory and analgesic drugs for treating chronic rheumatoid arthritis, arthritis deformans; or the use for manufacturing anti-inflammatory and analgesic drugs for treating low back pain, neck pain, cervico-omo-brachial syndrome, and periarthritis humeroscapularis; or the use for manufacturing anti-inflammatory and analgesic drugs for treating inflammation and pain caused by trauma. Secondly, use of a mixture of R-type optical isomer or its pharmaceutical acceptable salts and S-type optical isomer or its pharmaceutical acceptable salts with a ratio of 10:1-1:10 by weight for manufacturing anti-inflammatory and analgesic drugs, wherein a mixture with a ratio of 1:1 is excluded, including the use for manufacturing anti-inflammatory and analgesic drugs for treating chronic rheumatoid arthritis, arthritis deformans; or the use for manufacturing anti-inflammatory and analgesic drugs for treating low back pain, neck pain, cervico-omo-brachial syndrome, and periarthritis humeroscapularis; or the use for manufacturing anti-inflammatory and analgesic drugs for treating inflammation and pain caused by trauma.

The ratios (by weight) of R-type optical isomer to S-type optical isomer in the mixture are preferably 8:1, 6:1, 4:1, 2:1, 1:2, 1:4 or 1:6.

Said prepared anti-inflammatory and analgesic drugs are various formulations obtained by processing R-type optical isomer and a mixture having different ratio of R-type to S-type optical isomers with medical carriers, respectively, said formulations are selected from oral formulations such as tablets, capsules or oral liquids, topical formulations, delayed release formulations, or injectable formulations such as small volume injections or sterile injectable powders etc., preferably injectable formulations.

### DESCRIPTION OF FIGURES

FIG. 1: The effect of RS, S, R on toes' swelling of rats caused by carrageenin (n=8).

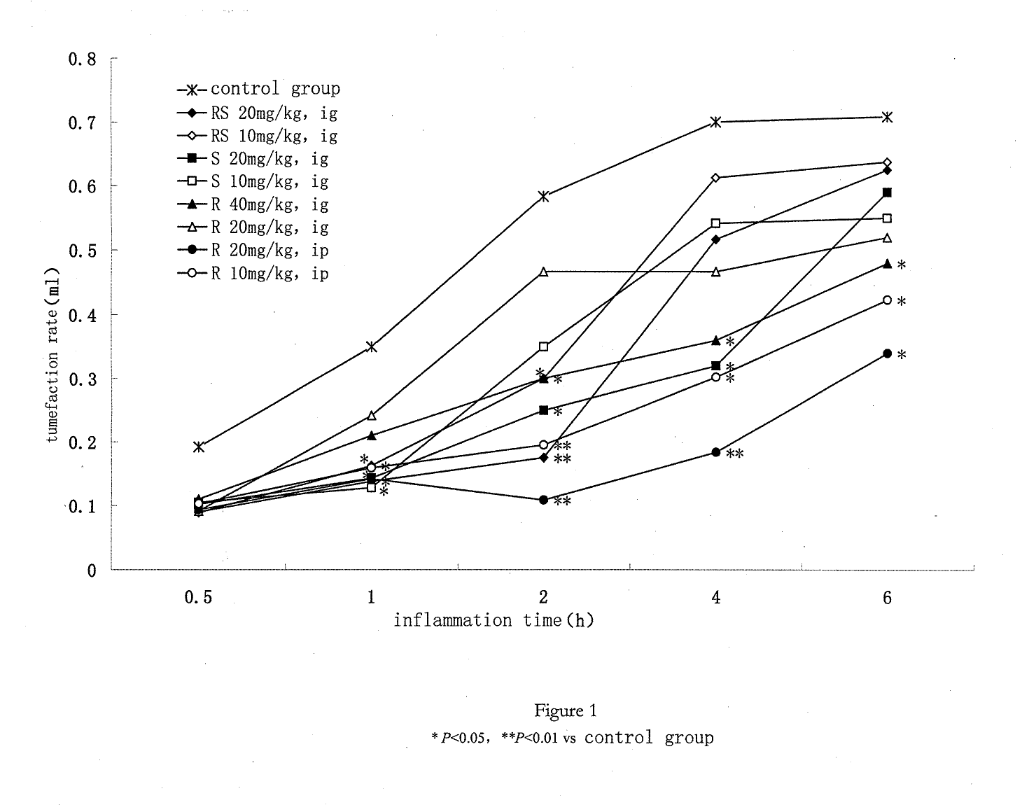

### PARTICULAR EMBODIMENTS

The following examples are provided to further describe the present invention.

<1> the tests of anti-inflammatory and analgesic effect in vitro of R-type, S-type, mixtures of RS with unequal ratios, and racemic 10,11-dihydro-alpha-methyl-10-oxo-dibenzo[b,f]tiazem-2-acetic acid.

1. The Purpose of the Tests

The mechanisms and characters of the anti-inflammatory and analgesic effects of RS, S-type, R-type 10,11-dihydro-alpha-methyl-10-oxo-dibenzo[b,f]tiazem-2-acetic acid can be illustrated by monitoring their inhibiting effects on arachidonic acid metabolism related enzymes such as epoxidase-1 (Cox-1), epoxidase-2 (Cox-2) and 5-lipoxidase (5-Lox), etc. Then, a comparison on the activities of S-type isomer and R-type isomer with different ratios are made.

2. Materials and Methods

2.1 Tested Compounds

RS (racemate), S-type, R-type 10,11-dihydro-alpha-methyl-10-oxo-dibenzo[b,f]tiazem-2-acetic acid, which are all white powders, their purities >98%. Just before tests, the required concentrations of stock solution are formulated with dimethyl sulfoxide (DMSO) ready-for-use.

2.2 Main Reagents and Materials

96-well microplates: Corning, USA

Cox-1: Cayman, USA

Cox-2: Cayman, USA

5-Lox (human recombinant): Cayman, USA

TMPD: Cayman, USA

Assay Buffer: Cayman, USA

Arachidonic Acid: Cayman, USA

NS-398: Cayman, USA

SC-560: Cayman, USA

Zileuton: Cayman, USA

Hematin: Sigma, USA

Developing Reagent: Cayman, USA

Colorimetric substrate: Cayman, USA

2.3 Main Apparatus

Centrifuger (Sigma, German)

Micropipette (Bio-Rad, USA)

Fluorescence Spectrometer (Safire2, Switzerland)

2.4 Tested Concentrations

Tested compounds and corresponding positive control compounds as well as their tested concentration are provided in the following table:

| Tested compounds | Concentrations (μmol/L) |
|---|---|
| NS-398 | 100, 30, 10, 3 |
| SC-560 | 100, 30, 10, 3 |
| Zileuton | 100, 30, 10, 3 |
| RS | 300, 100, 30, 10 |
| S | 300, 100, 30, 10 |
| R | 300, 100, 30, 10 |
| S:R = 8:1 | 300, 100, 30, 10 |
| S:R = 6:1 | 300, 100, 30, 10 |
| S:R = 4:1 | 300, 100, 30, 10 |
| S:R = 2:1 | 300, 100, 30, 10 |
| S:R = 1:2 | 300, 100, 30, 10 |
| S:R = 1:4 | 300, 100, 30, 10 |
| S:R = 1:6 | 300, 100, 30, 10 |
| S:R = 1:8 | 300, 100, 30, 10 |

2.5 Test Methods

The test procedures are provided as follows:

2.5.1 The Inhibiting Activity to COX-1/COX-2

(1) In 220 ul of reaction system, mixed buffer (1×), heme, COX-1/COX-2, and the compound to be tested, are sequentially added, and are incubated for 5 minutes at 25° C.;

(2) Adding chromogenic substrate (TMPD) and arachidonic acid;

(3) Carefully shaking the plate for several seconds, then incubating for 15 minutes at 25° C.;

(4) Detecting absorption value at 590 nm.

2.5.2 The Inhibiting Activity to 5-Lipoxygenase (5-Lox)

(1) In 100 ul of reaction system, mixed buffer (1×), substrate, 5-Lox, and the compound to be tested, are sequentially added, and are incubated for 5 minutes at 25° C.;

(2) Adding 100 μl of testing buffer into blank wells;

(3) After completion of the reaction, adding chromophore into each well to develop, and carefully shaking the plate for several seconds, then incubating for 15 minutes at 25° C.;

(4) Reading the plate at 500 nm to detect absorption value.

2.6 Data Calculation

Measuring the absorbance of each well, then calculating inhibition rate at each concentration=(absorption value of negative control−absorption value of sample to be tested)/(absorption value of negative control−absorption value of positive control)×100%. Fitting data of concentration (μmol/L) and corresponding inhibition rate (%) via Microsoft EXCEL software to get a trendline, so as to obtain the half-effective inhibiting concentration (IC50) of each tested enzyme.

3. Results

IC50 of inhibiting effects of each tested compound on Cox-1, Cox-2 and 5-Lox are provided in the following Table 1.

TABLE 1
the inhibiting effects of RS, S-type, R-type on Cox-1, Cox-2, and 5-Lox

| Tested | | IC50 (μmol/L) | | |
|---|---|---|---|---|
| compounds | concentrations (μmol/L) | Cox-1 | Cox-2 | 5-Lox |
| Positive control* | 100, 30, 10, 3 | 45.3 | 12.3 | 21.6 |
| RS | 300, 100, 30, 10 | 153 | 7.6 | 25.6 |
| S | 300, 100, 30, 10 | 186.2 | 21.6 | 49.2 |
| R | 300, 100, 30, 10 | >300 | 48.6 | 75.2 |
| S:R = 8:1 | 300, 100, 30, 10 | 173.6 | 9.3 | 43.6 |
| S:R = 6:1 | 300, 100, 30, 10 | 95.6^{#} | 5.2^{#} | 22.0^{#} |
| S:R = 4:1 | 300, 100, 30, 10 | 55.3^{#} | 4.1^{#} | 19.2^{#} |
| S:R = 2:1 | 300, 100, 30, 10 | 60.3^{#} | 14.5 | 30.9 |
| S:R = 1:2 | 300, 100, 30, 10 | 81.3^{#} | 6.2^{#} | 29.6 |
| S:R = 1:4 | 300, 100, 30, 10 | 109.5^{#} | 10.0 | 20.6^{#} |
| S:R = 1:6 | 300, 100, 30, 10 | 70.3^{#} | 5.2^{#} | 18.6^{#} |
| S:R = 1:8 | 300, 100, 30, 10 | 199.0 | 29.5 | 36.9 |

*Cox-1: SC-560,
Cox-2: NS-398,
5-Lox:Zileuton;
^{#}< RS

The results show that RS, S, and R have the effects of inhibiting Cox-1, Cox-2, and 5-Lox to different degrees, wherein the inhibiting effects on Cox-2 are the highest. The order of the inhibition strength on the enzymes is: RS>S>R. Furthermore, in the samples with other ratios (S:R), the samples, whose inhibition strength is higher than those of RS (1:1), include: 6:1, 4:1, 1:2, 1:4, 1:6.

4. Conclusion

RS has the effects of inhibiting Cox-1, Cox-2, and 5-Lox to different degrees, wherein the inhibiting effect on Cox-2 is the highest, and its inhibiting effect on three kinds of enzymes are higher that those of S- and R-. As a whole, in the samples with other ratios (S:R), the inhibiting effects of the samples at ratios of 6:1, 4:1, 1:2, 1:4, 1:6, etc. are higher than the effects of RS (1:1).

<2> the tests of anti-inflammatory and analgesic effect in vivo of R-type, S-type, RS mixture with different proportions, and racemic 10,11-dihydro-alpha-methyl-10-oxo-dibenzo[b,f]tiazem-2-acetic acid.

1. The Purpose of the Tests:

The purposes of the tests include: observing and studying anti-inflammatory and analgesic effects in vivo of RS, S-, R-; making a comparison on strengths or characters of three kinds of samples; and performing the pharmacodynamic comparative tests on the S-type isomer and R-type isomer with different proportions.

2. Test Materials

2.1 Tested Compounds

RS (racemate), S-type, R-type 10,11-dihydro-alpha-methyl-10-oxo-dibenzo[b,f]tiazem-2-acetic acid, which are all white powders, and their purities >98%. Just before tests, a stock solution with desired concentration is formulated with dimethyl sulfoxide (DMSO) ready-for-use.

2.2 Apparatus and Reagents

2.2.1 Apparatus

FEJ-200 electronic balance (0.1-200 g), Fuzhou Furi Weighing Electronics Co., Ltd.

BS210S precision electronic balance (0.1 mg˜10 g), Sartorius, Germany

SYC super intelligence thermostatic waterbath, Gongyi Yingyuyuhua Instrument Plant

2.2.2 Main Reagents

Xylene, Nanjing Chemical Reagent Co., Ltd, analytical pure, batch number: 071211192;

0.9% Sodium Chloride Injection, Nanjing Xiaoying drug manufacturer, batch number: 2007020602;

λ-carrageen (C3889, Lot 122k1444), Sigma Company;

Acetic acid (glacial acetic acid), analytical pure, Nanjing Chemical Reagent Co., Ltd, batch number: 071011006;

2.3 Animals:

ICR Mus musculus albus, 18-22 g, clean grade, purchased from the center of experimental animals of Nantong University, Certificate Number of the animals: SCXK (Su) 2003-0002.

ICR Mus musculus albus, 18-22 g, clean grade, purchased from the center of comparative medicine of Yangzhou University, Certificate Number of the animals: SCXK (Su) 2007-0001.

SD rats, 150-180 g, SPF Grade, purchased from the center of experimental animals of academy of military medical sciences, Certificate Number of the animals: SCXK (Jun) 2007-0004.

3. Test Methods and Results

3.1 The Effect on Writhing in Mice Induced by Acetic Acid

3.1.1 Dosages and Grouping

On the basis of preliminary test, 370 healthy Kunming species of Mus musculus albus are selected, male, weight 18-22 g, and totally 13 tested groups, corresponding to RS, S-type, R-type and the S- and R-mixtures with unequal ratios, respectively. Except for the control group, each tested group includes 3 dose-groups, and each dose-group includes 10 mice, said tested group comprising:

(1) blank control group, 0.5% CMC—Na, 0.1 ml/10 g, ig

(2) RS, (30, 10, 3) mg/kg, 0.1 ml/10 g, ig

(3) S, (30, 10, 3) mg/kg, 0.1 ml/10 g, ig

(4) R, (90, 30, 10) mg/kg, 0.1 ml/10 g, ig

(5) S:R=8:1 (30, 10, 3) mg/kg, 0.1 ml/10 g, ig

(6) S:R=6:1 (30, 10, 3) mg/kg, 0.1 ml/10 g, ig

(7) S:R=4:1 (30, 10, 3) mg/kg, 0.1 ml/10 g, ig

(8) S:R=2:1 (30, 10, 3) mg/kg, 0.1 ml/10 g, ig

(9) S:R=1:1 (30, 10, 3) mg/kg, 0.1 ml/10 g, ig

(10) S:R=1:2 (30, 10, 3) mg/kg, 0.1 ml/10 g, ig

(11) S:R=1:4 (30, 10, 3) mg/kg, 0.1 ml/10 g, ig

(12) S:R=1:6 (30, 10, 3) mg/kg, 0.1 ml/10 g, ig

(13) S:R=1:8 (30, 10, 3) mg/kg, 0.1 ml/10 g, ig

wherein RS is a synthetic racemate, S:R=1:1 is milled mixture at a ratio of 1:1 of S-type isomer to R-type isomer. For each group, the required weights of drugs are weighted, and are milled with 0.5% CMC—Na to required concentration ready-for-use.

3.1.2 Induction of Pain and Administration Method

Each group of animals is fasted for 12 h, but drinking ad libitum. At 1 hour after intragastric administration of corresponding drugs, 0.6% acetic acid (0.2 ml/per) is intraperitoneally injected, and the number of writhing of mice in 15 min after injection are recored. The mice with waist muscle repeat shrinkage, bump back, rump torsion and posterior limb stretch are used as writhing positive animals. Inhibition rates of drugs on writhing are calculated based on the following formula:

Inhibition rate (%)=[(number of writhing of the control group−number of writhing of administrated group)/number of writhing of the control group]×100%

3.1.3 Indexes of Evaluation

Inhibition rate of each dose-group on writhing (%) is obtained by means of above method. Fitting data of dosage (mg/kg) and corresponding inhibition rate (%) via Microsoft EXCEL software to get a trendline, so as to obtain the half-effective inhibiting dose (ED50) of each tested group on writhing.

3.1.4 Results

Compared with the blank control group (0.5% CMC—Na), high dose-group and middle dose-group of RS, S-, R- and each formulated group can all significantly inhibit writhing in mice induced by acetic acid (P<0.05, P<0.01), and details are omitted. The maximum inhibition rate of ED50 of each tested group on writhing is provided in the following Table 2.

TABLE 2
The effect of ig administration of each tested group on mouse writhing
(n = 10, x̅ ± s)|

| | Groups | | maximum | |
|---|---|---|---|---|
| Numbers | (mg/kg) | Dosages | inhibitory rate* (%) | ED50 (mg/kg) |
| A | RS | 30, 10, 3 | 63.6 | 9.2 |
| B | S | 30, 10, 3 | 80.1 | 8.6 |
| C | R | 90, 30, 10 | 71.3 | 25.4 |
| D | S:R = 8:1 | 30, 10, 3 | 76.2 | 8.0 |
| E | S:R = 6:1 | 30, 10, 3 | 86.2 | 7.1 |
| F | S:R = 4:1 | 30, 10, 3 | 97.5 | 6.3 |
| G | S:R = 2:1 | 30, 10, 3 | 92.5 | 6.6 |
| H | S:R = 1:1 | 30, 10, 3 | 66.9 | 9.1 |
| I | S:R = 1:2 | 30, 10, 3 | 86.6 | 7.1 |
| J | S:R = 1:4 | 30, 10, 3 | 80.7 | 7.6 |
| K | S:R = 1:6 | 30, 10, 3 | 88.2 | 6.9 |
| L | S:R = 1:8 | 30, 10, 3 | 57.7 | 10.6 |

*The maximum inhibitory rate of three tested groups.

3.1.5 Conclusions

It can be seen from Table 1 that maximum inhibition rate and ED50 for RS racemate and R-, S-mixture at a ratio of 1:1 are comparable. Based on ED50 value, the order of effect strengths of above each tested sample is: F>G>K>I≈E>J>D>B>H≈A(RS)>L>C.

3.2 The Effect on Hot Plate Pain Threshold of Mice

3.2.1 Dosages and Grouping

On the basis of preliminary test, 90 healthy Kunming species of Mus musculus albus are selected, female, weight 18˜22 g, totally 8 tested groups, and each group of 10 mice, said tested groups comprising:

(1) blank control group, 0.5% CMC—Na, 0.1 ml/10 g, ig

(2) RS, 80 mg/kg, 0.1 ml/10 g, ig

(3) RS, 40 mg/kg, 0.1 ml/10 g, ig

(4) S, 80 mg/kg, 0.1 ml/10 g, ig

(5) S, 40 mg/kg, 0.1 ml/10 g, ig

(6) R, 160 mg/kg, 0.1 ml/10 g, ig

(7) R, 80 mg/kg, 0.1 ml/10 g, ig

(8) R, 40 mg/kg, 0.1 ml/10 g, ig

(9) R, 40 mg/kg, 0.1 ml/10 g, ip

3.2.2 Induction of Pain and Administration Method

Each group of animals is pasted for 12 h, but drinking ad libitum. Mice are placed on a homoiothermal hot plate (55±0.5□), and the time of occurrence of licking foot is recorded as pain threshold. Appropriate mice (whose reaction time of pain is less than 60 seconds) are selected and randomly divided into groups, and pain thresholds are measured twice before administration and the average value is used as base pain threshold. At 0.5 h and 1 hour after intragastric administration of corresponding drugs, the pain threshold of each mouse is measured (if there are no reaction of pain in 60 seconds, then the threshold is considered as 60 second).

3.2.3 Statistical Analysis

Using Microsoft EXCEL software, the average value of pain threshold of each tested group and the average value of control group are performed t-test analysis, and P<0.05 is considered as statistically significant difference.

3.2.4 Results

As shown in Table 3, compared with control group, once 0.5 h and 1 h after administration of R 160 mg/kg (ig) and R 40 mg/kg (ip), pain threshold of mice can be significantly prolonged (P<0.05), and other tested groups had no significant effects on hot plate pain threshold of mice.

TABLE 3
The effect of ig administration of RS, S-, R-on the hot plate pain
threshold of mice
(n = 10, x̅ ± s)|

| | Dosages | pain threshold(s) | | |
|---|---|---|---|---|
| Groups | (mg/kg) | 0 h | 0.5 h | 1 h |
| Control | 0.1 ml/10 g | 16.6 ± 5.3 | 16.3 ± 6.9 | 15.8 ± 6.4 |
| group | | | | |
| (0.5% | | | | |
| CMC-Na) | | | | |
| RS | 80, ig | 12.7 ± 3.1 | 11.7 ± 5.2 | 9.6 ± 3.5 |
| RS | 40, ig | 12.8 ± 3.7 | 9.9 ± 2.7 | 10.7 ± 5.7 |
| S | 80, ig | 13.6 ± 9.6 | 12.7 ± 6.9 | 7.9 ± 1.8 |
| S | 40, ig | 12.9 ± 5.0 | 10.8 ± 2.7 | 10.6 ± 4.6 |
| R | 160, ig | 22.0 ± 4.9 | 24.1 ± 15.1* | 27.3 ± 11.9* |
| R | 80, ig | 21.5 ± 15.5 | 17.5 ± 15.7 | 17.5 ± 12.3 |
| R | 40, ig | 17.6 ± 13.8 | 12.0 ± 7.8 | 14.8 ± 9.9 |
| R | 40, ip | 15.2 ± 15.1 | 20.0 ± 15.7* | 23.7 ± 20.5* |

*P < 0.05, vs control group.

3.2.5 Conclusions

At 0.5 h and 1 h after administration of R 160 mg/kg (ig) and R 40 mg/kg (ip), pain threshold of mice can be significantly prolonged (P<0.05), but the highest dose-group of RS and S, i.e. 80 mg/kg (ig) dose-group, has no significant effect on hot plate pain threshold of mice.

3.3 The Effect on Auricle Tumefaction of Mice Caused by Xylene

3.3.1 Dosages and Modeling Method

90 healthy Kunming species of Mus musculus albus are selected, male, weight 18˜22 g, totally 9 tested groups, and each group of 10 mice, said tested groups comprising:

(1) blank control group, 0.5% CMC—Na, 0.1 ml/10 g, ig

(2) RS, 20 mg/kg, 0.1 ml/10 g, ig

(3) RS, 10 mg/kg, 0.1 ml/10 g, ig

(4) S, 20 mg/kg, 0.1 ml/10 g, ig

(5) S, 10 mg/kg, 0.1 ml/10 g, ig

(6) R, 80 mg/kg, 0.1 ml/10 g, ig

(7) R, 40 mg/kg, 0.1 ml/10 g, ig

(8) R, 40 mg/kg, 0.1 ml/10 g, ip

(9) R, 20 mg/kg, 0.1 ml/10 g, ip

At 0.5 h before modeling, groups (8) and (9) are administrated tested drugs, and other groups are administrated tested drugs at 1 h before modeling. During modeling, the right ears of mice are coated with xylene at concentration of 30 μl/per ear to inflame the ear, and the left ears are performed no treatment. Once 3 h after coating xylene, the animals are sacrificed, and two ears of each mouse are cut, and ear wafers are obtained by making holes at the same positions of ears with puncher with diameter of 8 mm, and then are weighted via an electronic balance.

3.3.2 Indexes of Detection

The tumefaction degree refers to the weight difference of the weight of inflamed ear wafer minus non-inflamed ear wafer. The tumefaction rate tumefaction degree/the weight of normal ear wafer×100‰ The average value of the tumefaction degree and tumefaction rate of each tested group is compared, so as to obtain the ability of anti-inflammation of each tested drug.

3.3.3 Statistical Analysis

Using Microsoft EXCEL software, the average value of tumefaction degree of each tested group and the average value of control group are analyzed by means of t-test, and P<0.05 is considered as statistically significant difference.

3.3.4 Results

The results can be seen in the following Table 4. In 3 h after inducing inflammation, the each dose-group has effects of inhibition to auricle tumefaction to different degrees.

TABLE 4
The effects of RS, R-, and S-on auricle tumefaction of mice caused
by xylene
(n = 10, x̅ ± s)|

| | | tumefaction |
|---|---|---|
| Groups | Dosages (mg/kg) | degree (mg) |
| blank control group | 0.1 ml/10 g, ig | 6.6 ± 3.2 |
| (0.5% CMC-Na) | | |
| RS | 20, ig | 3.7 ± 2.5* |
| RS | 10, ig | 5.2 ± 2.9 |
| S | 20, ig | 3.1 ± 3.2* |
| S | 10, ig | 4.9 ± 2.3 |
| R | 80, ig | 2.5 ± 1.4** |
| R | 40, ig | 3.8 ± 2.8* |
| R | 40, ip | 2.3 ± 1.8** |
| R | 20, ip | 3.5 ± 2.0* |

*P < 0.05,
**P < 0.01 vs control group

3.3.5 Conclusions

When ig administration, RS is comparable to R in improving auricle tumefaction; R 80 mg/kg, 40 mg/kg are better than RS 20 mg/kg, 10 mg/kg respectively in inhibiting tumefaction; R 40 mg/kg is comparable to RS 20 mg/kg in its strength of effect; R ip administration is better than R ig administration in inhibiting tumefaction.

3.4 The Effect on Toes' Swelling of Rats Caused by Carrageenin

3.4.1 Dosages and Modeling Method

90 healthy SD rats are selected, male, weight 150˜180 g, totally 9 tested groups, and each group of 8 rats, said tested groups comprising:

(1) blank control group, 0.5% CMC—Na, 0.5 ml/100 g, ig

(2) RS, 20 mg/kg, 0.5 ml/100 g, ig

(3) RS, 10 mg/kg, 0.5 ml/100 g, ig

(4) S, 20 mg/kg, 0.5 ml/100 g, ig

(5) S, 10 mg/kg, 0.5 ml/100 g, ig

(6) R, 40 mg/kg, 0.5 ml/100 g, ig

(7) R, 20 mg/kg, 0.5 ml/100 g, ig

(8) R, 20 mg/kg, 0.5 ml/100 g, ip

(9) R, 10 mg/kg, 0.5 ml/100 g, ip

The animals of groups (1)-(7) are intragastric administrated (ig) drugs, at 1 h before they are injected carrageen to induce inflammation, and the animals of groups (8)-(9) are intraperitoneally injected (ip) drugs, at 0.5 h before they are injected carrageen to induce inflammation.

3.4.2 The Induction of Toes' Swelling of Rats

On one day before test, 1% carrageen is formulated, and is weighted in a certain ratio, then is placed into 0.9% sodium chloride injection to swell overnight ready to use. The animals before induction of inflammation, are administrated 1 ml/100 g of distilled water via intragastric administration, and then the hind limbs of the animals are stretched, and in the middle of right toes 0.1 ml of 1% carrageen is injected to induce inflammation.

3.4.3 Indexes of Detection

The device is self-made according to related reference documents, and the toes' volume of rat is measured via displacement of water. The volume is measured once just after injection of carrageen as base volume. Then at 0.5, 1, 2, 4, and 6 h after induction of inflammation, the volume of hind foot is measured, and the time points of swelling reaching maximum and swelling regression are observed and recorded, so as to calculate tumefaction degree and tumefaction rate at each time point. The difference between administrated groups and control groups is compared, so as to calculate the inhibition rate of drugs on swelling, so as to evaluate the anti-inflammatory effect. The formula is provided as follows:

tumefaction degree (ml)=(measured volume−base volume)

tumefaction rate (%)=[(measured volume−base volume)/base volume]×100%

3.4.4 Statistical Analysis

Using Microsoft EXCEL software, the average value of tumefaction degree of each tested group and the average value of control group are analyzed by means of t-test, and P<0.05 is considered as statistically significant difference.

3.4.5 Results

After carrageen are injected into the toe of rat, its hind foot swelled gradually, and the swelling reaches the maximum at 2-4 h after injection, then the swelling slowly regressed after 6 h. The results of tests show that each dose-group of three samples RS, S, R improves tumefaction degrees in rats to different degrees in 6 h after inflammation induced by carrageen. Compared with the blank control group, the groups and time points having statistically significant improvement are provided as follows:

(1) RS 20 mg/kg, ig: 1 h (P<0.05), 2 h (P<0.01)

(2) RS 10 mg/kg, ig: 1 h (P<0.05), 2 h (P<0.05)

(3) S 20 mg/kg, ig: 1 h (P<0.05), 2 h (P<0.05), 4 h (P<0.05)

(4) S 10 mg/kg, ig: 1 h (P<0.05)

(5) R 40 mg/kg, ig: 2 h (P<0.05), 4 h (P<0.05), 6 h (P<0.05)

(6) R 40 mg/kg, ig: for each time points P>0.05

(7) R 20 mg/kg, ip: 1 h (P<0.05), 2 h (P<0.01), 4 h (P<0.01), 4 h (P<0.05)

(8) R 10 mg/kg, ip: 1 h (P<0.05), 2 h (P<0.01), 4 h (P<0.05), 4 h (P<0.05)

3.4.6 Conclusions

When ig administration, each of RS 20 mg/kg, S 20 mg/kg, and R 40 mg/kg could significantly improve toes' swelling in rats induced by carrageen, and their strengths of effect are comparable. Further, ip administration of R 20 mg/kg had a significant improvement to toes' swelling, better than those of ig administration of same doses of RS, S and R.

4. Summary

The analgesic effects of various samples RS, S, R are tested by writhing in mice induced by acetic acid and hot plate method.

The results of mouse writhing response showed that the analgesic effects of various samples RS, S, R are significant, the ED50 (mg/kg) of inhibition of writhing response by RS, S, R are: 9.2, 8.6, 25.4, respectively, which showed that order of analgesic strengths is: S>RS>R, wherein effect strength of R is about ⅓ of that of RS.

Based on the evaluated criteria of writhing in mice induced by acetic acid, the analgesic effects of samples comprising different ratios of S and R are tested, and the analgesic effects are compared with those of RS, and the results showed the order of effect strengths [S:R (ED50, mg/kg)] is: 4:1 (6.3)>2:1 (6.6)>1:6 (6.9)>1:2 (7.1)≈6:1 (7.1)>1:4 (7.6)>8:1 (8.0)>S (8.6)>1:1 (9.1)≈RS (9.2)>1:8 (10.6)>R (25.4). Noted that the effects of the samples with S:R=4:1, 2:1, 1:6, 1:2, 6:1, 1:4 and 8:1 are all better than that of RS (S:R=1:1).

Hot plate tests in mice showed that at 0.5 h and 1 h after administration (ip) of R 160 mg/kg (ig) and R 40 mg/kg, pain thresholds of mice can be significantly prolonged (P<0.05), and R 80 mg/kg (ig) had a certain range of improvement. However, the highest dose-group of RS and S, 80 mg/kg (ig) dose-group, had no significant effect on hot plate pain threshold of mice, which suggested that R had a better improvement on hot plate pain threshold in mice that S and RS.

The anti-inflammatory effects of various samples RS, S, R are tested by the models of auricle tumefaction of mice caused by xylene and toes' swelling of rats caused by carrageenin.

The results of model of mice using xylene showed when ig administration, the improvements of RS and S to auricle tumefaction are comparable, and the inhibitions of R 80 mg/kg, 40 mg/kg on tumefaction are better than RS 20 mg/kg, 10 mg/kg, respectively, and the strength of effect of R 40 mg/kg is comparable to that of RS 20 mg/kg; when ip administration of R, the inhibition of administration is better than that of ig administration. The results suggested that the inhibition of RS on tumefaction is better than that of R, and the strength of the former is about as twice as the latter.

The results of model of rats using carrageen showed when ig administration, each of RS 20 mg/kg, S 20 mg/kg, and R 40 mg/kg could significantly improve toes' swelling in rats induced by carrageen, and their strengths of effect are comparable. Further, ip administration of R 20 mg/kg had a significant improvement to toes' swelling, better than those of ig administration of same doses of RS, S and R. The results suggested that the inhibition of RS on swelling is better than that of R, and the strength of the former is about as twice as the latter.

The results of the present studies showed that: (1) the various samples RS, S, R had significantly anti-inflammatory and analgesic effects, and as a whole, the order of strengths of effect is S≧RS>R, wherein effect strength of R is about ½-⅓ of that of RS; (2) ip administration of R had significantly better anti-inflammatory and analgesic effects than ig administration of same dose of R, and better than ig administration of same dose of RS and S; and (3) the effects of drugs with S:R=4:1, 2:1, 1:6, 1:2, 6:1, 1:4, 8:1 are all better than that of RS (S:R=1:1).

<3> The acute toxicity test in mice of ig administration of R-type, S-type, unequal ratio mixtures of RS, and racemic 10,11-dihydro-alpha-methyl-10-oxo-dibenzo[b,f]tiazem-2-acetic acid

1. The Purpose of the Tests

Measuring LD₅₀ of acute toxicity value in mice ig administrated RS, S, R to learn the main toxic symptom.

2. Materials and Methods

2.1 Tested Drugs

RS (racemate), S-type, R-type 10,11-dihydro-alpha-methyl-10-oxo-dibenzo[b,f]tiazem-2-acetic acid, whose appearances are all white powders, and their purities each >98%. Just before tests, the required concentrations of stock solution are formulated with dimethyl sulfoxide (DMSO) ready-for-use.

2.2 Animals

(1) Source: ICR mice, purchased from the center of comparative medicine of Yangzhou University, Certificate Number of the animals: SCXK (Su) 2007-0001.

(2) Weight: 18-22 g

(3) Gender: Fifty-fifty of male and female

(4) The periods of fasting: 12 h, drinking ad libitum.

(5) Numbers of animals in each group: 10

2.3 Dosages and Grouping

2.3.1 Dosage: Based on the results of preliminary test, totally 12 tested groups are prepared, corresponding to RS, S, R as well as the unequal ratio mixtures of S and R, respectively. Each tested group includes 5 dose-groups during the tests as follows:

(1) RS: 400, 340, 289, 245.6, 208.8 mg/kg;

(2) S: 400, 340, 289, 245.6, 208.8 mg/kg;

(3) R: 5000, 4250, 3612, 3070, 2610 mg/kg;

(4) S:R=8:1: 400, 340, 289, 245.6, 208.8 mg/kg;

(5) S:R=6:1: 400, 340, 289, 245.6, 208.8 mg/kg;

(6) S:R=4:1: 400, 340, 289, 245.6, 208.8 mg/kg;

(7) S:R=2:1: 400, 340, 289, 245.6, 208.8 mg/kg;

(8) S:R=1:1: 400, 340, 289, 245.6, 208.8 mg/kg;

(9) S:R=-1:2: 750, 637.5, 541.9, 460.6, 391.5 mg/kg;

(10) S:R=1:4: 1000, 850, 722.5, 614.1, 522.0 mg/kg;

(11) S:R=1:6: 1500, 1275, 1083.8, 921.2, 783.0 mg/kg;

(12) S:R=1:8: 2000, 1700, 1445, 1228.3, 1044.0 mg/kg;

2.3.2 Interval of Administration: 1:0.85

2.3.3 The Amount Administrated: 0.4 ml/20 g

2.4 Administration Route

Intragastric administration (ig)

2.5 Test Methods

(1) The environments of laboratory: room temperature of 24±2° C., relative humidity of 60-70%.

(2) Observed indexes: respectively formulating drug suspensions of above-described 5 dosages in series dilutions of RS, S, R based on the administrated volumes; ig administrating equal volume of drugs once; recording each toxic symptoms of mice and amounts of dead mice; and autopsying the dead animals.

(3) Observed periods: 14 days

3. Results

3.1 The Observation of General Condition

After ig administration of RS, S, R, reduction of spontaneous action, weight loss, lying on their stomachs, ptosis, slow response to exoteric stimulus, and quiet death in each of mice could be seen. On the day when drugs are administrated, no animals are dead, and the deaths of animals mainly occurred on 2-3 days after administration, and the deaths of animals still existed in some tested groups on day 4 after administration, and no animals are dead on day 5 after administration. In groups of RS and S, the actions of alive animals begin to increase and returned back to normal after 5 days, whereas the degrees of change of general conditions and spontaneous actions of animals in R groups are less than those of RS and S groups, and recovered faster. With increasing of ratio of R in each groups, the effect of drugs on spontaneous action in each group tended to decrease.

3.2 The Results of Autopsying

In the observation of autopsying dead cases, stomach swelling, and aqueous-like or white contents in gastrointestinal tract could be seen. Dead mice are observed by naked eye, and no significant lesions are observed in their viscera. After administration, it is possible for mice to affect their gastrointestinal tracts and inhibit nerve centers, which finally resulted in systemic failure to dead.

3.3 LD₅₀ Value of ig Administration of RS, S, R in Mice

To calculate according to Bliss method, and LD₅₀ value of ig administration of various samples of RS, S, R in mice are measured and are provided in Table 5. The results showed that when S:R≧1.0, LD₅₀ values for samples formulated in each ratio are relatively close, and all are 300˜400 mg/kg; and when S:R≦1.0, LD₅₀ values for samples formulated in each ratio increased with increasing of ratio of R in each drug.

TABLE 5
LD₅₀ value of ig administration of RS, S, R in mice

| | | | | Com. Vs. |
|---|---|---|---|---|
| Numbers | Groups | Dosages (mg/kg) | LD₅₀ (mg/kg) | S (multiple) |
| 1 | RS | 400, 340, 289, 245.6, 208.8 | 326.9 ± 30.7 | 1.2 |
| 2 | S | 400, 340, 289, 245.6, 208.8 | 267.9 ± 22.2 | 1.0 |
| 3 | R | 5000, 4250, 3612, 3070, 2610 | 3993.9 ± 153.0 | 14.9 |
| 4 | S:R = 8:1 | 400, 340, 289, 245.6, 208.8 | 368.2 ± 29.6 | 1.4 |
| 5 | S:R = 6:1 | 400, 340, 289, 245.6, 208.8 | 326.5 ± 32.6 | 1.2 |
| 6 | S:R = 4:1 | 400, 340, 289, 245.6, 208.8 | 359.5 ± 40.3 | 1.3 |
| 7 | S:R = 2:1 | 400, 340, 289, 245.6, 208.8 | 332.5 ± 45.6 | 1.2 |
| 8 | S:R = 1:1* | 400, 340, 289, 245.6, 208.8 | 375.6 ± 33.9 | 1.4 |
| 9 | S:R = 1:2 | 750, 637.5, 541.9, 460.6, 391.5 | 578.3 ± 41.3 | 2.2 |
| 10 | S:R = 1:4 | 1000, 850, 722.5, 614.1, 522.0 | 802.3 ± 78.6 | 3.0 |
| 11 | S:R = 1:6 | 1500, 1275, 1083.8, 921.2, 783.0 | 1138.5 ± 80.3 | 4.2 |
| 12 | S:R = 1:8 | 2000, 1700, 1445, 1228.3, 1044.0 | 1675.3 ± 102.6 | 6.3 |

*milled mixture of 1:1 of S-type isomer to R-type isomer after resolution.

4. Conclusions

When mice are ig administrated drugs, LD₅₀ values of R (3993.9±153.0 mg/kg) is far more than that of S (267.9±22.2 mg/kg). In the formulations with different ratios of S and R, when S:R≧1.0, LD₅₀ values for samples formulated in each ratio are relatively close, and all are 300˜400 mg/kg; and when S:R≦1.0, LD₅₀ values for samples formulated in each ratio increased with increasing of ratio of R in each drug. It is suggested that the acute toxicity of ig administration of S is far more than that of R, when the ratio between R and S is S:R≦1.0, R could mitigate dose-dependently the acute toxicity of ig administration of S.

<4> The acute toxicity test in mice of ip administration of R-type, S-type, unequal ratio mixtures of RS, and racemic 10,11-dihydro-alpha-methyl-10-oxo-dibenzo[b,f]tiazem-2-acetic acid

1. The Purpose of the Tests

Measuring LD₅₀ value of acute toxicity value in mice ip administrated RS, S, R to learn the main toxic symptom.

2. Materials and Methods

2.1 Tested Drugs

RS (racemate), S-type, R-type 10,11-dihydro-alpha-methyl-10-oxo-dibenzo[b,f]tiazem-2-acetic acid, whose appearances are all white powders, and their purifies each >98%. Just before tests, the required concentrations of stock solution are formulated with dimethyl sulfoxide (DMSO) ready-for-use.

2.2 Animals

(1) Source: ICR mice, purchased from the center of comparative medicine of Yangzhou University, Certificate Number of the animals: SCXK (Su) 2007-0001

(2) Weight: 18-22 g

(3) Gender: Fifty-fifty of male and female

(4) The periods of fasting: No fasting.

(5) Numbers of animals in each group: 10.

2.3 Dosages and Grouping

2.3.1 Dosage: Based on the results of preliminary test, totally 12 tested groups are prepared, corresponding to RS, S, R as well as the unequal ratio mixtures of S and R, respectively. Each tested group includes 5 dose-groups during the tests as follows:

(1) RS: 300, 255, 216.8, 184.2, 156.6 mg/kg;

(2) S: 300, 255, 216.8, 184.2, 156.6 mg/kg;

(3) R: 1000.0, 850.0, 722.5, 614.1, 522.0 mg/kg;

(4) S:R=8:1: 300, 255, 216.8, 184.2, 156.6 mg/kg;

(5) S:R=6:1: 300, 255, 216.8, 184.2, 156.6 mg/kg;

(6) S:R=4:1: 300, 255, 216.8, 184.2, 156.6 mg/kg;

(7) S:R=2:1: 300, 255, 216.8, 184.2, 156.6 mg/kg;

(8) S:R=1:1: 300, 255, 216.8, 184.2, 156.6 mg/kg;

(9) S:R=1:2: 300, 255, 216.8, 184.2, 156.6 mg/kg;

(10) S:R=1:4: 400, 340, 289, 245.6, 208.8 mg/kg;

(11) S:R=1:6: 600, 510, 433.5, 368.5, 313.2 mg/kg;

(12) S:R=1:8: 800, 680, 578, 491.3, 417.6 mg/kg.

2.3.2 Interval of Administration: 1:0.85

2.3.3 The Amount Administrated: 0.4 ml/20 g

2.4 Administration Route

Intraperitoneal injection (ip)

2.5 Test Methods

(1) The environments of laboratory: room temperature of 24±2° C., relative humidity of 60-70%.

(2) Observed indexes: respectively formulating drug suspensions of above-described 5 dosages in series dilutions of RS, S, R based on the administrated volumes; ip administrating equal volume of drugs once; recording each toxic symptoms of mice and amounts of dead mice; and autopsying the dead animals.

(3) Observed periods: 14 days.

3. Results

3.1 The Observation of General Condition

After ip administration of RS, S, R, reduction of spontaneous action, weight loss, lying on their stomachs, ptosis, slow response to exoteric stimulus, and quiet death in each of mice could be seen. On the day when drugs are administrated, deaths of animals are observed, and the deaths of animals mainly occurred on 2-3 days after administration, and the deaths of animals still existed in some tested groups on day 4 after administration, and no animals are dead on day 5 after administration. In groups of RS and S, the actions of alive animals began to increase and returned back to normal after 5 days, whereas the degrees of change of general conditions and spontaneous actions of animals in R groups are less than those of RS and S groups, and recovered faster. With increasing of ratio of R in each groups, the effect of drugs on spontaneous action in each group tended to decrease.

3.2 The Results of Autopsying

Dead mice are observed by naked eye, and no significant lesions are observed in their viscera. After administration, it is possible for mice to affect their gastrointestinal tracts and inhibit nerve centers, which finally resulted in systemic failure to dead.

3.3 LD₅₀ Value of ip Administration of RS, S, R in Mice

To calculate according to Bliss method, and LD₅₀ value of ip administration of various samples of RS, S, R in mice are measure, and are provided in Table 6. The results showed that when S:R≧1.0, LD₅₀ values for samples formulated in each ratio are relatively close, and all are 200˜300 mg/kg; and when S:R≦1.0, LD₅₀ values for samples formulated in each ratio increased with increasing of ratio of R in each drug.

TABLE 5
LD₅₀ value of ip administration of RS, S, R in mice

| | | | | Com. Vs. |
|---|---|---|---|---|
| Numbers | Groups | Dosages (mg/kg) | LD₅₀ (mg/kg) | S (multiple) |
| 1 | RS | 300, 255, 216.8, 184.2, 156.6 | 252.2 ± 18.9 | 1.3 |
| 2 | S | 300, 255, 216.8, 184.2, 156.6 | 200.8 ± 14.3 | 1.0 |
| 3 | R | 1000, 850, 722.5, 614.1, 522.0 | 744.2 ± 69.8 | 3.7 |
| 4 | S:R = 8:1 | 300, 255, 216.8, 184.2, 156.6 | 201.6 ± 16.8 | 1.0 |
| 5 | S:R = 6:1 | 300, 255, 216.8, 184.2, 156.6 | 220.3 ± 18.6 | 1.1 |
| 6 | S:R = 4:1 | 300, 255, 216.8, 184.2, 156.6 | 213.6 ± 17.6 | 1.1 |
| 7 | S:R = 2:1 | 300, 255, 216.8, 184.2, 156.6 | 235.5 ± 20.3 | 1.2 |
| 8 | S:R = 1:1* | 300, 255, 216.8, 184.2, 156.6 | 249.0 ± 19.5 | 1.2 |
| 9 | S:R = 1:2 | 300, 255, 216.8, 184.2, 156.6 | 245.2 ± 19.6 | 1.2 |
| 10 | S:R = 1:4 | 400, 340, 289, 245.6, 208.8 | 319.4 ± 20.3 | 1.6 |
| 11 | S:R = 1:6 | 600, 510, 433.5, 368.5, 313.2 | 497.6 ± 28.6 | 2.5 |
| 12 | S:R = 1:8 | 800, 680, 578, 491.3, 417.6 | 615.4 ± 38.9 | 3.1 |

*milled mixture of 1:1 of S-type isomer to R-type isomer after resolution.

4. Conclusions

When mice are ip administrated drugs, LD₅₀ values of R (744.2±69.8 mg/kg) is more than that of S (200.8±14.3 mg/kg). In the formulations with different ratios of S and R, when S:R≧1.0, LD₅₀ values for samples formulated in each ratio are relatively close, and all are 200˜300 mg/kg; and when S:R≦1.0, LD₅₀ values for samples formulated in each ratio increased with increasing of ratio of R in each drug. It is suggested that the acute toxicity of ip administration of S is more than that of R, when the ratio between R and S is S:R≦1.0, R could mitigate dose-dependently the acute toxicity of ip administration of S.

<5> The toxicity tests in SD rats subjected four-week consecutive intragastric administration of Zaltoprofen

1. the purpose of the tests: the studies of long-term toxicity test of ig administration of various Zaltoprofen samples in rodents, SD rats, are performed in this test; the various responses of SD rats subjected four-week consecutive intragastric administration are observed; target organs of toxic reaction and reversibility of damage to rats are determined; the dosages of nontoxic reaction are determined; and the toxicity of racemate are compared with those of single isomer, as well as mixed samples having isomers with different ratios (RS, R, S, R:S=6:1) so as to evaluate the safety of long-term administration of various Zaltoprofen samples so as to provide a reference for the dosage used by human.

2. Test methods: total 4 tested samples are prepared, which are racemate Zaltoprofen (RS, 80, 40, 20 mg/kg), R-Zaltoprofen (R, 480, 240, 120 mg/kg), S-Zaltoprofen (S, 80, 40, 20 mg/kg), R-Zaltoprofen:S-Zaltoprofen=6:1 (6R1S, 320, 160, 80 mg/kg), respectively, and an additional blank control group is prepared; totally 13 tested groups, 10 rats in each tested group, and all fifty-fifty of male and female. The tested samples are formulated into suspensions by milling with 0.5% CMC—Na; intragastric administration every day (10 ml/kg) for consecutive 4 weeks; on each day, after administration, the general conditions of the animals are observed, and weight and amount of ingestion are determined once every week. In 24 hours after the last administration, the rats are fasted overnight, blood samples are withdrawn via femoral arteries, and the rats are dissected, and the indexes of blood cytology and biochemics, gloss anatomy and histopathological examination are preformed.

3. Results: during 4 weeks administration, 2 animals (male) are dead in racemate Zaltoprofen group, 80 mg/kg, and four animals (3 male, 1 female) and one animal (male) are dead in S-Zaltoprofen 80 and 40 mg/kg groups, respectively, no other death are observed in each group. Severe seroperitoneum and gastrointestinal tract perforation in dissected animals are observed; histopathological examinations of dead animals showed ablation, degeneration, and necrosis of intestinal mucous membrane, as well as many inflammatory effusion. Except dead animals, no significant abnormalities related to drugs are found out by observing the general conditions of alive animals.

Compared with the blank control group, weight gains of the rats subjected to high dose-group of each tested drug and in S-Zaltoprofen dose-group are inhibited to the different extents, in rest tested drugs, no significant abnormalities of weights, weight gains or ingestion amounts are observed in comparison with the blank control group.

The results of hematological examinations show that main indexes changed in each tested group included that numbers of erythrocytes in higher dose-group partly decreased, and numbers of leucocytes in same groups partly increased, and numbers of hemoblasts significantly increased in comparison with the blank control group, and there are no significant differences among tested groups of respective samples.

The results of serological biochemical examinations show that main indexes changed in each tested group include that the concentrations of blood sugar and uric acid partly decreased in comparison with the blank control group, and there are no significant difference among tested groups of respective samples.

The results of organ coefficient measurements showed that no significant changes in ease tested group are observed in comparison with the blank control group, and there are no significant difference among tested groups of respective samples.

The results of histopathological examination show that in alive animals in each tested group, changes such as eosinophilic degeneration of partial liver, inflammatory infiltration of intestinal tract, degeneration of bladder epithelium and the like could be observed in comparison with the blank control group; and incidence of the changes is low and most of changes do not have dose-effect relationship; and there are no significant difference among tested groups of respective samples.

4. Conclusions: under the conditions of the test, long-term administration of high dose of S-Zaltoprofen (80, 40 mg/kg) and RS-Zaltoprofen (80 mg/kg) produces significant gastrointestinal tract toxicity, and results in animals dead, whereas each dose-group of R-Zaltoprofen (480, 240, 120 mg/kg) and the mixtures with R:S=6:1 (320, 160, 80 mg/kg) do not produce significant gastrointestinal tract toxicity. By combining the results of hematological, serological biochemical, histopathological examinations, it can be seen that the main toxic target organs of each of Zaltoprofen samples are gastrointestinal tracts, and toxic degree has a significant dose-dependent property. The results of this test show that nontoxic dosages of RS, R, S, R:S=6:1 in the rats subjected four-week consecutive intragastric administration are 40, 240, 20, and 160 mg/kg, respectively. Based on this criterion, the safety of R-Zaltoprofen and mixture with R:S=6:1 are six times or more and four times or more of that of RS-Zaltoprofen, respectively.

<6> The long-term toxicity tests in SD rats subjected to thirteen-week consecutive intragastric administration of Zaltoprofen and four-week convalescence

1. The purpose of the tests: the studies of long-term toxicity test of ig administration of various Zaltoprofen samples in rodents, SD rats, are performed in this test; the various responses of SD rats subjected to thirteen-week consecutive intragastric administration and withdrawn four-week for recovery are observed; target organs of toxic reaction and reversibility of damage to rats are determined; the dosages of nontoxic reaction are determined; and the toxicity of racemate are compared with those of single isomer, as well as mixed samples having different ratios of isomers (RS, R, S, R:S=6:1), so as to evaluate the safety of long-term administration of various Zaltoprofen samples, so as to provide a reference for the dosage used by human.

2. Test methods: total 4 tested samples are prepared, which are racemate (RS, 50, 25, 12.5 mg/kg), R-Zaltoprofen (R, 400, 200, 100 mg/kg), S-Zaltoprofen (S, 50, 25, 12.5 mg/kg), R-Zaltoprofen:S-Zaltoprofen=6:1 (6R1S, 200, 100, 50 mg/kg), respectively, and an additional blank control group is prepared; totally 13 tested groups, 20 rats in each tested group, and all fifty-fifty of male and female. The tested samples are formulated into suspensions by milling with 0.5% CMC—Na; intragastric administration (10 ml/kg) every day for consecutive 13 weeks; on each day, after administration, the general conditions of the animals are observed, and weight and amount of ingestion are determined once every week. In 24 hours after the last administration, half of rats in each tested group are fasted overnight, blood samples are withdrawn via femoral arteries, and the rats are dissected, and the indexes of blood cytology and biochemics, gloss anatomy and histopathological examination are preformed. The administration to remaining half rats is stopped, and the convalescence is observed; general conditions are observed and weight and amount of ingestion are determined once every week. After 4 weeks, the observation is complete, and the half of rats in each tested group are fasted overnight, blood samples are withdrawn via femoral arteries, and the rats are dissected, and the indexes of blood cytology and biochemics, gloss anatomy and histopathological examination are preformed.

3. Results:

3.1 The examined results of thirteen-week consecutive intragastric administration during thirteen-week administration, 2 animals (female) are dead in racemate Zaltoprofen group, 50 mg/kg, and 2 animals (female) and 1 animal (female) are dead in S-Zaltoprofen 50 and 20 mg/kg groups, respectively, no other death are observed in each group. Severe seroperitoneum and gastrointestinal tract perforation in dissected animals are observed; histopathological examinations of dead animals showed ablation, degeneration, and necrosis of intestinal mucous membrane, as well as many inflammatory effusion. Except dead animals, no significant abnormalities related to drugs are found out by observing the general conditions of alive animals.

Compared with the blank control group, weight gains of the rats subjected to high dose-group of each tested drug and in S-Zaltoprofen dose-group are inhibited to the different extents, in rest tested drugs, no significant abnormalities of weights, weight gains or ingestion amounts are observed in comparison with the blank control group.

The results of hematological examinations show that main indexes changed in each tested group include that numbers of erythrocytes in higher dose-group partly decreased, and numbers of leucocytes in same groups partly increased, and numbers of hemoblasts significantly increased in comparison with the blank control group, and there are no significant difference among tested groups of respective samples.

The results of serological biochemical examinations show that main indexes changed in each tested group include the concentrations of blood sugar and uric acid partly decreased in comparison with the blank control group, and there are no significant difference among tested groups of respective samples.

The results of organ coefficient measurements showed that no significant changes in ease tested group are observed in comparison with the blank control group, and there are no significant difference among tested groups of respective samples.

The results of histopathological examination showed that in alive animals in each tested group, changes such as eosinophilic degeneration of partial liver, inflammatory infiltration of intestinal tract, degeneration of bladder epithelium and the like could be observed in comparison with the blank control group; and incidence of the changes is low and most of changes did not have dose-effect relationship; and there are no significant difference among tested groups of respective samples.

3.2 The Observed Results of Convalescence

The observed results of convalescence showed that after stopping administrating, the general conditions, weights, and ingested amounts of each group of tested drugs are similar with those of blank control group, and no significant changes related to drugs are observed. At the end of convalescence, the results of hematological, serological biochemical, histopathological examinations show that no significant changes in ease tested group are observed in comparison with the blank control group, and there are no significant difference among tested groups of respective samples.

4. Conclusions:

Under the conditions of the test, long-term administration of high dose of S-Zaltoprofen (50, 25 mg/kg) and RS-Zaltoprofen (50 mg/kg) produces significant gastrointestinal tract toxicity, and results in animals dead, whereas each dose-group of R-Zaltoprofen (400, 200, 100 mg/kg) and the mixtures with R:S=6:1 (200, 100, 50 mg/kg) does not result in part of animals dead, and not produce significant gastrointestinal tract toxicity, and in low and middle dose-group, none of indexes shows significant toxic changes related to tested drugs. The results of this test show that the main toxic target organs of each of Zaltoprofen samples are gastrointestinal tracts, toxic degree has a significant dose-dependent property, and the changes caused by most drugs can be recovered after drug withdrawal, i.e., reversible. With combination of each tested index, nontoxic dosages of RS, R, S, R:S=6:1 in the rats subjected to 13-week consecutive intragastric administration are 25, 200, 12.5, and 100 mg/kg, respectively. Based on this criterion, the safeties of R-Zaltoprofen and R:S=6:1 mixture of 13-week consecutive intragastric administration of rats are eight times or more and four times or more of that of RS-Zaltoprofen, respectively.

<7> The comparison of therapeutic indexes of R-type, S-type, an unequal ratio mixture of RS, and racemate 10,11-dihydro-alpha-methyl-10-oxo-dibenzo[b,f]tiazem-2-acetic acid on writhing response in mice induced by acetic acid via writhing response in mice induced by acetic acid, ED50 of analgesic effect of intragastric administration (ig) of RS, S, R is determined, at same time, LD₅₀ of ig administration in mice is determined, thereby, therapeutic index=LD₅₀/ED50, and data are provided in flowing Table 7.

TABLE 7
The therapeutic index of IG administration of RS, S, R

| | | | therapeutic |
|---|---|---|---|
| Groups | ED50 (mg/kg) | LD₅₀ (mg/kg) | index |
| RS | 9.2 | 326.9 | 35.5 |
| S | 8.6 | 267.9 | 31.2 |
| R | 25.4 | 3993.9 | 157.2 |

Results show that: therapeutic index of R are higher than those of RS and S.

<8> The comparison of therapeutic indexes of an unequal ratio mixture of RS, and racemate 10,11-dihydro-alpha-methyl-10-oxo-dibenzo[b,f]tiazem-2-acetic acid on writhing response in mice induced by acetic acid

Via writhing response in mice induced by acetic acid, ED₅₀ of analgesic effect of intragastric administration (ig) of samples formulated with RS, S:R=4:1, 2:1, 1:6, 1:2, 6:1, 1:4, 8:1 is determined, at same time, LD₅₀ of ig administration in mice is determined, thereby, therapeutic index=LD₅₀/ED₅₀, and data are provided in flowing Table 8.

TABLE 8
The therapeutic index of ig administration of mixture having different
ratios of R and S

| Groups | ED50 (mg/kg) | LD₅₀ (mg/kg) | therapeutic index |
|---|---|---|---|
| RS | 9.2 | 326.9 | 35.5 |
| S:R = 8:1 | 8.0 | 318.2 | 39.8 |
| S:R = 6:1 | 7.1 | 306.5 | 43.2 |
| S:R = 4:1 | 6.3 | 359.5 | 57.1 |
| S:R = 2:1 | 6.6 | 332.5 | 50.4 |
| S:R = 1:2 | 7.1 | 578.3 | 81.5 |
| S:R = 1:4 | 7.6 | 802.3 | 105.6 |
| S:R = 1:6 | 6.9 | 1138.5 | 165.0 |

<9> Pharmacokinetic studies in rats of ig administration of R-type 10,11-dihydro-alpha-methyl-10-oxo-dibenzo[b,f]tiazem-2-acetic acid

Pharmacokinetic studies in rats of ig administration of R-type 10,11-dihydro-alpha-methyl-10-oxo-dibenzo[b,f]tiazem-2-acetic acid shows that: ig pharmacokinetic behavior of R-type optical isomer is well, and corresponds to two-compartment model, and there are certain linear relationship among plasma drug concentration, AUC and dosage. After intragastric administration, the drugs are introduced into blood and distributed quickly, and the elimination of drug is relatively slow. No transformation between R-isomer and S-isomer is detected during the monitoring of blood drug level. Data are provided in flowing Table 7.

TABLE 9
Main parameters of pharmacokinetics after
ig administration of 50 mg/kg R-type isomer in rats

| Parameters | units | A | B | C | D | E | F | Mean | SD |
|---|---|---|---|---|---|---|---|---|---|
| AUC(0-48) | mg/L*h | 723.9 | 239.0 | 330.6 | 814.3 | 324.2 | 1336.0 | 628.0 | 418.9 |
| AUG(0-∞) | mg/L*h | 772.8 | 256.2 | 337.7 | 818.7 | 325.6 | 1374.1 | 647.5 | 430.3 |
| Cmax | mg/L | 260.8 | 98.9 | 67.8 | 223.1 | 131.5 | 184.2 | 161.1 | 74.5 |
| Tmax | h | 1.0 | 1.0 | 0.5 | 0.3 | 0.3 | 0.5 | 0.6 | 0.3 |
| t1/2z | h | 10.0 | 2.2 | 9.8 | 7.8 | 4.9 | 9.6 | 7.4 | 3.2 |
| MRT(0-48) | h | 7.4 | 2.2 | 9.1 | 5.4 | 5.0 | 10.3 | 6.6 | 3.0 |
| CLz/F | L/h/kg | 0.1 | 0.2 | 0.1 | 0.1 | 0.2 | 0.0 | 0.1 | 0.1 |
| Vz/F | L/kg | 0.9 | 0.6 | 2.1 | 0.7 | 1.1 | 0.5 | 1.0 | 0.6 |

<10> The preparation of R-10,11-dihydro-alpha-methyl-10-oxo-dibenzo[b,f]tiazem-2-acetic acid tablet

Formulation (1000 tablets):

| | | |
|---|---|---|
| R-type isomer | 80.0 | g |
| lactose | 80.0 | g |
| microcrystalline cellulose | 24.0 | g |
| sodium carboxymethyl starch | 12.0 | g |
| polyvinyl pyrrolidone | 2.4 | g |
| magnisium stearate | 0.24 | g |
| Products | 1000 | tablets |

Preparation method: R-10,11-dihydro-alpha-methyl-10-oxo-dibenzo[b,f]tiazem-2-acetic acid, lactose, and microcrystalline cellulose are weighted, and then sufficiently uniformly mixed. After polyvinyl pyrrolidone is dissolved with 25 ml of 50% ethanol, the solution is added to above mixed powder, to prepare damp mass. The damp mass is passed through 24-mesh to form wet particles, which then is dried for 3 hours at 60□ and normal pressure to obtain dry particles. The dry particles are passed through 24-mesh to granulate, and sodium carboxymethyl starch and magnesium stearate are added, and uniformly mixed. The mixed powders are pressed into tablets weighting 200 mg using a punch with a diameter of 8.0 mm.

<11> The preparation of freeze-dried powder injector for injection of R-10,11-dihydro-alpha-methyl-10-oxo-dibenzo[b,f]tiazem-2-acetic acid

Formulation (1000 injectors):

| R-type isomer | 80.0 | g |
|---|---|---|
| Mannitol | 80.0 | g |
| 0.1M sodium hydroxide | pH 3.0~5.0 | |
| Water for injection | to total 2000 | ml |
| Products | 1000 | injectors |

Preparation method: R-10,11-dihydro-alpha-methyl-10-oxo-dibenzo[b,f]tiazem-2-acetic acid, lactose, and mannitol are dissolved into appropriated amount of water for injection, and 0.1M sodium hydroxide is added to dissolve the main ingredient and to adjust the pH to pH 3.0˜5.0, and then the water for injection is added to total volume of 2 L. 0.1% active carbon is added to decolor the mixture, and after pyrogen removal, the active carbon are filtered out. The filtration sterilization of liquid medicine is performed, followed by aseptically filling and freeze-drying to obtain freeze-dried powder injector for injection with 80 mg/ampoule.

<12> The preparation of sodium salt of R,S-10,11-dihydro-alpha-methyl-10-oxo-dibenzo[b,f]tiazem-2-acetic acid

Preparation method: in reaction flask, R,S-10,11-dihydro-alpha-methyl-10-oxo-dibenzo[b,f]tiazem-2-acetic acid (15 g, 0.05 mol) is added to aqueous solution of sodium hydroxide (100 ml, 0.5M), and after dissolution via stirring, 5 volumes of solution of methanol is added to deposit out white solid, left overnight, then filtered to obtain white solid which is vacuum-dried at 60° C. to obtain the sodium salt of 12.5 g R,S-10,11-dihydro-alpha-methyl-10-oxo-dibenzo[b,f]tiazem-2-acetic acid.

<13> The preparation of potassium salt of R,S-10,11-dihydro-alpha-methyl-10-oxo-dibenzo[b,f]tiazem-2-acetic acid

Preparation method: see example 12 for preparation method

<14> The preparation of calcium salt of R,S-10,11-dihydro-alpha-methyl-10-oxo-dibenzo[b,f]tiazem-2-acetic acid

Preparation method: in reaction flask, R,S-10,11-dihydro-alpha-methyl-10-oxo-dibenzo[b,f]tiazem-2-acetic acid (15 g, 0.05 mol) is added, then aqueous solution calcium chloride (200 ml, 0.025M) in 40% ethanol is added, then the mixture is heated to reflux to dissolve all the solids, followed by cooled overnight to deposit out white solid which is dried and filtered to obtain 9.5 g of white-like solid.

<15> The preparation of sodium salt of R-10,11-dihydro-alpha-methyl-10-oxo-dibenzo[b,f]tiazem-2-acetic acid

Preparation method: see example 12 for preparation method

<16> The preparation of potassium salt of R-10,11-dihydro-alpha-methyl-10-oxo-dibenzo[b,f]tiazem-2-acetic acid

Preparation method: see example 12 for preparation method

<17> The preparation of calcium salt of R-10,11-dihydro-alpha-methyl-10-oxo-dibenzo[b,f]tiazem-2-acetic acid

Preparation method: see example 14 for preparation method

<18> The preparation of sodium salt of S-10,11-dihydro-alpha-methyl-10-oxo-dibenzo[b,f]tiazem-2-acetic acid

Preparation method: see example 12 for preparation method

<19> The preparation of potassium salt of S-10,11-dihydro-alpha-methyl-10-oxo-dibenzo[b,f]tiazem-2-acetic acid

Preparation method: see example 12 for preparation method

<20> The preparation of calcium salt of S-10,11-dihydro-alpha-methyl-10-oxo-dibenzo[b,f]tiazem-2-acetic acid

Preparation method: see example 14 for preparation method

<21> The preparation of sodium salt of R-10,11-dihydro-alpha-methyl-10-oxo-dibenzo[b,f]tiazem-2-acetic acid:S-10,11-dihydro-alpha-methyl-10-oxo-dibenzo[b,f]tiazem-2-acetic acid (6:1)

Preparation method: see example 12 for preparation method

<22> The preparation of potassium salt of R-10,11-dihydro-alpha-methyl-10-oxo-dibenzo[b,f]tiazem-2-acetic acid:S-10,11-dihydro-alpha-methyl-10-oxo-dibenzo[b,f]tiazem-2-acetic acid (6:1)

Preparation method: see example 12 for preparation method

<22> The preparation of calcium salt of R-10,11-dihydro-alpha-methyl-10-oxo-dibenzo[b,f]tiazem-2-acetic acid:S-10,11-dihydro-alpha-methyl-10-oxo-dibenzo[b,f]tiazem-2-acetic acid (6:1)

Preparation method: see example 14 for preparation method

<23> The preparation of L-arginine salt of R,S-10,11-dihydro-alpha-methyl-10-oxo-dibenzo[b,f]tiazem-2-acetic acid

To R, S-10,11-dihydro-alpha-methyl-10-oxo-dibenzo[b,f]tiazem-2-acetic acid (5 g, 0.01675 mol), L-arginine (2.919 g, 0.01675 mol) is added, then 150 ml of 95% ethanol is added. The mixture is then refluxed for 1 h, left at room temperature overnight while stirring, to deposit out jelly-like solid which is filtered, and the solid is collected, washed with ethanol, and dried over phosphorus pentoxide under vacuum at 60° C. to obtain about 6 g of yellow solid.

<24> The preparation of L-arginine salt of R-10,11-dihydro-alpha-methyl-10-oxo-dibenzo[b,f]tiazem-2-acetic acid

R-10,11-dihydro-alpha-methyl-10-oxo-dibenzo[b,f]tiazem-2-acetic acid (4 g, 0.0134 mol) is dissolved in 100 ml of ethanol at 50-60° C., then L-arginine (2.3352 g, 0.0134 mol) and 40 ml of aqueous solution of 50% ethanol are dropwise added, and while dropwise adding, deposition of solid occurred. After addition, the mixture is stirring at 50-60° C. for 1 h, left at room temperature overnight, to deposit out a lot of white solid which is filtered, washed with ethanol, and the solid is collected and dried over phosphorus pentoxide under vacuum at 60° C. to obtain 5.28 g of white solid.

<25> The preparation of L-arginine salt of S-10,11-dihydro-alpha-methyl-10-oxo-dibenzo[b,f]tiazem-2-acetic acid

S-10,11-dihydro-alpha-methyl-10-oxo-dibenzo[b,f]tiazem-2-acetic acid (4 g, 0.0134 mol) is dissolved in 160 ml of ethanol under refluxing, then L-arginine (2.3352 g, 0.0134 mol) and 40 ml of aqueous solution of 50% ethanol are dropwise added, and while dropwise adding, no deposition of solid occurred. After addition, the mixture is refluxed for 30 minutes, and then stirred at room temperature and left overnight, to deposit out jelly-like solid which is filtered, washed with ethanol, and dried over phosphorus pentoxide under vacuum at 60° C. to obtain about 3.78 g of white solid.

<26> The preparation of L-arginine salt of R-10,11-dihydro-alpha-methyl-10-oxo-dibenzo[b,f]tiazem-2-acetic acid:S-10,11-dihydro-alpha-methyl-10-oxo-dibenzo[b,f]tiazem-2-acetic acid (6:1)

R-10,11-dihydro-alpha-methyl-10-oxo-dibenzo[b,f]tiazem-2-acetic acid:S-10,11-dihydro-alpha-methyl-10-oxo-dibenzo[b,f]tiazem-2-acetic acid (6:1) (4 g, 0.0134 mol) is dissolved in 100 ml of ethanol at 50-60° C., then L-arginine (2.3352 g, 0.0134 mol) and 40 ml of aqueous solution of 50% ethanol are dropwise added, and while dropwise adding, deposition of solid occurred. After addition, the mixture is stirring at 50-60° C. for 1 h, left at room temperature overnight, to deposit out white solid which is filtered, washed with ethanol, and the solid is collected and dried over phosphorus pentoxide under vacuum at 60° C. to obtain 4.32 g of light yellow solid.

<27> The preparation of L-lysine salt of R,S-10,11-dihydro-alpha-methyl-10-oxo-dibenzo[b,f]tiazem-2-acetic acid

To R, S-10,11-dihydro-alpha-methyl-10-oxo-dibenzo[b,f]tiazem-2-acetic acid (3 g, 0.01005 mol), L-lysine (1.4688 g, 0.01005 mol) is added, and then 100 ml of 95% ethanol is added. The mixture is then refluxed for 1 h. If deposition occurred, the deposit could be filtered out. The filtrate is concentrated under reduced pressure to dryness while stirring, to obtain yellow solid.

<28> The preparation of L-lysine salt of R-10,11-dihydro-alpha-methyl-10-oxo-dibenzo[b,f]tiazem-2-acetic acid

To R-10,11-dihydro-alpha-methyl-10-oxo-dibenzo[b,f]tiazem-2-acetic acid (3 g, 0.01005 mol), L-lysine (1.4688 g, 0.01005 mol) is added, and then 60 ml of 95% ethanol is added. The mixture is then refluxed for 1 h to deposit out solid, and is left at room temperature to deposit out a lot of solid which is filtered, washed with ethanol, and dried at 60° C. under vacuum, to obtain 2.31 g of yellow-like solid.

<29> The preparation of L-lysine salt of S-10,11-dihydro-alpha-methyl-10-oxo-dibenzo[b,f]tiazem-2-acetic acid

To S-10,11-dihydro-alpha-methyl-10-oxo-dibenzo[b,f]tiazem-2-acetic acid (3 g, 0.01005 mol), L-lysine (1.4688 g, 0.01005 mol) is added, and then 100 ml of 95% ethanol is added. The mixture is then refluxed for 1 h. If deposition occurred, the deposit could be filtered out. The filtrate is concentrated under reduced pressure to dryness while stirring, to obtain yellow solid.

<30> The preparation of L-lysine salt of R-10,11-dihydro-alpha-methyl-10-oxo-dibenzo[b,f]tiazem-2-acetic acid:S-10,11-dihydro-alpha-methyl-10-oxo-dibenzo[b,f]tiazem-2-acetic acid (6:1)

R-10,11-dihydro-alpha-methyl-10-oxo-dibenzo[b,f]tiazem-2-acetic acid:S-10,11-dihydro-alpha-methyl-10-oxo-dibenzo[b,f]tiazem-2-acetic acid (6:1) (4 g, 0.0134 mol) is dissolved in 100 ml of ethanol at 50-60° C., then L-lysine (2.3352 g) and 40 ml of aqueous solution of 50% ethanol are dropwise added, and while dropwise adding, deposition of solid occurred. After addition, the mixture is stirring at 50-60° C. for 1 h, left at room temperature overnight, to deposit out solid which is filtered, washed with ethanol, and the solid is collected and dried over phosphorus pentoxide under vacuum at 60° C. to obtain 3.34 g of light yellow solid.

<31> The preparation of R-10,11-dihydro-alpha-methyl-10-oxo-dibenzo[b,f]tiazem-2-acetic acid

In there-necked flask, (R, S)-Zaltoprofen (117 g, 0.3926 mol) is added, and then 700 ml of methanol is added to dissolve the solid. The mixture is heated to reflux under which the mixed solution of D-(+)-α-phenylethylamine (38.88 g, 0.3213 mol) and 100 ml of methanol is dropwise added. After the mixture is stirred for 2 h at room temperature, a lot of solid is deposited out, which is filtered to obtain crude product of R-10,11-dihydro-alpha-methyl-10-oxo-dibenzo[b,f]tiazem-2-acetic acid-D-(+)-α-phenylethylamine salt as light yellow, and filtered cake is washed with methanol.

To above obtained crude product of R-10,11-dihydro-alpha-methyl-10-oxo-dibenzo[b,f]tiazem-2-acetic acid-D-(+)-α-phenylethylamine salt, 500 ml of methanol is added, and refluxed. The mixture is left overnight at room temperature, and a lot of white solid is deposited out, which is refined product of resolved salt.

240 ml of aqueous solution of 0.1 mol/L sodium hydroxide is added to and dissolve the refinedly resolved salt, and the mixture is extracted with 120 ml of ethyl ether twice. To the aqueous phase, aqueous solution of 0.1 mol/L hydrochloric acid is added to acidify the solution to PH 3, and the mixture is extracted with ethyl acetate thrice. The three extracted liquids are combined, and solvent is vaporized under reduced pressure, and residues is recrystallized with ethanol to obtain 24 g of R-10,11-dihydro-alpha-methyl-10-oxo-dibenzo[b,f]tiazem-2-acetic acid.

<32> The preparation of S-10,11-dihydro-alpha-methyl-10-oxo-dibenzo[b,f]tiazem-2-acetic acid

In there-necked flask, (R, S)-Zaltoprofen (80 g, 0.2684 mol) is added, and then 470 ml of methanol is added to dissolve the solid. The mixture is heated to reflux under which the mixed solution of L-(−)-α-phenylethylamine (26 g, 0.2148 mol) and 70 ml of methanol is dropwise added. After completion of addition, the reflux reaction is carried out for 30 minutes. The mixture is then stirred for 2 h at room temperature, a lot of solid is deposited out, which is filtered to obtain crude product of S-10,11-dihydro-alpha-methyl-10-oxo-dibenzo[b,f]tiazem-2-acetic acid-L-(−)-α-phenylethylamine salt as light yellow, and filtered cake is washed with methanol.

To above obtained crude product of S-10,11-dihydro-alpha-methyl-10-oxo-dibenzo[b,f]tiazem-2-acetic acid-L-(−)-α-phenylethylamine, 370 ml of methanol is added, and refluxed for 30-40 minutes. The mixture is left overnight at room temperature, and a lot of white solid is deposited out, which is refined product of resolved salt.

160 ml of aqueous solution of 0.1 mol/L sodium hydroxide is added to and dissolve the refinedly resolved salt, and the mixture is extracted with 90 ml of ethyl ether twice. To the aqueous phase, aqueous solution of 0.1 mol/L hydrochloric acid is added to acidify the solution to PH 3 to deposit out a lot of solid, and the mixture is extracted with ethyl acetate thrice. The three extracted liquids are combined, and solvent is vaporized under reduced pressure, and residues is recrystallized with ethanol to obtain 14 g of S-10,11-dihydro-alpha-methyl-10-oxo-dibenzo[b,f]tiazem-2-acetic acid.

<33> The preparation of R-10,11-dihydro-alpha-methyl-10-oxo-dibenzo[b,f]tiazem-2-acetic acid:S-10,11-dihydro-alpha-methyl-10-oxo-dibenzo[b,f]tiazem-2-acetic acid (6:1)

R-10,11-dihydro-alpha-methyl-10-oxo-dibenzo[b,f]tiazem-2-acetic acid (6.000 g) and S-10,11-dihydro-alpha-methyl-10-oxo-dibenzo[b,f]tiazem-2-acetic acid (1.000 g) are accurately measured, and 100 ml of methanol is added. After dissolution via heating, the mixture is cooled, and then the methanol is vaporized under the reduced pressure to obtain 7 g of R-10,11-dihydro-alpha-methyl-10-oxo-dibenzo[b,f]tiazem-2-acetic acid:S-10,11-dihydro-alpha-methyl-10-oxo-dibenzo[b,f]tiazem-2-acetic acid (6:1) 7 g.

<34> Chromatographic condition determined by the use of C18 chromatographic column:

1. Chromatographic Condition

With octadecylsilane chemically bonded silica as a filler; methanol-water-glacial acetic acid (70:30:0.5) as mobile phase; flow rate as 1.0 ml/min; and detection wavelength as 254 nm.

2. The Formulation of Test Solution

6.25 mg of above test sample is accurately weighted, placed into 25 ml volumetric flask, dissolved with mobile phase, and diluted to the mark, shaken, filtered, and formulated into a solution have 0.25 mg pre 1 ml as test solution.

3. Measured Method

Accurate 20 ul of test solution is injected into liquid chromatograph, and the chromatogram is recorded, and calculated based on area normalization method.

<35> Chromatographic condition of R, S isomers determined by the use of chiral chromatographic column:

1. Chromatographic Condition

With Chiralcel OJ-RH 150*4.6 mm chiral column; 0.15 mol/l sodium perchlorate buffer (solution of 70% sodium perchlorate adjusted pH to pH 2.5):methanol (20:80) as mobile phase; flow rate as 0.4 ml/min; and detection wavelength as 220 nm.

2. The Formulation of Test Solution

12.5 mg of above test sample is accurately weighted, placed into 25 ml volumetric flask, dissolved with mobile phase, and diluted to the mark, shaken, filtered; accurate 1 ml of filtrate is withdrawn, and placed into 25 ml volumetric flask, diluted it to the mark with mobile phase, shaken, and formulated into a solution have 20 ug pre 1 ml as test solution.

3. Measured Method

Accurate 20 ul of test solution is injected into liquid chromatograph, and the chromatogram is recorded, and calculated based on area normalization method.

The foregoing contents further describe the present invention, however, the present invention is not limited the specific embodiments as described herein, and includes all modified forms which fall into the scope of attached claims.

1. A treatment method for treating one or more selected from inflammation, pain symptoms and diseases, wherein the method comprises administering to a subject in need of such treatment a therapeutically effective amount of R-type 10,11-dihydro-alpha-methyl-10-oxo-dibenzo[b,f]tiazem-2-acetic acid or its pharmaceutical acceptable salts, wherein the pharmaceutical acceptable salts are selected from sodium salts, potassium salts, calcium salts and amino acid salts.

2. The treatment method according to claim 1, wherein the diseases are selected from chronic rheumatoid arthritis, arthritis deformans, cervico-omo-brachial syndrome and periarthritis humeroscapularis.

3. The medical use treatment method according to claim 1, wherein the R-type 10,11-dihydro-alpha-methyl-10-oxo-dibenzo[b,f]tiazem-2-acetic acid or its pharmaceutical acceptable salts is used for manufacturing anti-inflammatory and analgesic drugs in the treatment of the pain symptoms are selected from low back pain, neck pain, and inflammation and pain which are caused by trauma.

4. The treatment method according to claim 1, wherein the administration is by an oral, external or injection route.

5. Optical isomers of phenylpropionic acid drugs, wherein said optical isomers of phenylpropionic acid drugs are a mixture having R-type 10,11-dihydro-alpha-methyl-10-oxo-dibenzo[b,f]tiazem-2-acetic acid or its pharmaceutical acceptable salts and S-type 10,11-dihydro-alpha-methyl-10-oxo-dibenzo[b,f]tiazem-2-acetic acid or its pharmaceutical acceptable salts with a ratio of 10:1-1:10 by weight, wherein the ratio of 1:1 is excluded.

6. The optical isomers of phenylpropionic acid drugs according to claim 5, wherein the ratio of R-type 10,11-dihydro-alpha-methyl-10-oxo-dibenzo[b,f]tiazem-2-acetic acid or its pharmaceutical acceptable salts to the S-type 10,11-dihydro-alpha-methyl-10-oxo-dibenzo[b,f]tiazem-2-acetic acid or its pharmaceutical acceptable salts in the mixture does not include 1:2, 1:4 and 1:9.

7. The optical isomers of phenylpropionic acid drugs according to claim 5, wherein the ratio of the R-type 10,11-dihydro-alpha-methyl-10-oxo-dibenzo[b,f]tiazem-2-acetic acid or its pharmaceutical acceptable salts to the S-type 10,11-dihydro-alpha-methyl-10-oxo-dibenzo[b,f]tiazem-2-acetic acid or its pharmaceutical acceptable salts is one selected from 8:1, 6:1, 4:1, 2:1 and 1:6.

8. The optical isomers of phenylpropionic acid drugs according to claim 5, wherein the ratio by weight of the R-type 10,11-dihydro-alpha-methyl-10-oxo-dibenzo[b,f]tiazem-2-acetic acid or its pharmaceutical acceptable salts to the S-type 10,11-dihydro-alpha-methyl-10-oxo-dibenzo[b,f]tiazem-2-acetic acid or its pharmaceutical acceptable salts in the mixture is one selected from 1:2 and 1:4.

9. A treatment method for treating one or more selected from inflammation, pain symptoms and diseases, wherein the method comprises administering to a subject in need of such treatment a therapeutically effective amount of the optical isomers of phenylpropionic acid drugs according to claim 5.

10-16. (canceled)

17. The treatment method according to claim 1, wherein the diseases are selected from chronic rheumatoid arthritis, arthritis deformans, cervico-omo-brachial syndrome and periarthritis humeroscapularis.

18. The treatment method according to claim 1, wherein the symptoms are selected from low back pain, neck pain, and inflammation and pain which are caused by trauma.

19. The treatment method according to claim 1, wherein the administration is by an oral, an external or an injection route.

20. A treatment method for treating one or more selected from inflammation, pain symptoms and diseases, wherein the method comprises administering to a subject in need of such treatment a therapeutically effective amount of the optical isomers of phenylpropionic acid drugs according to claim 6.

21. A treatment method for treating one or more selected from inflammation, pain symptoms and diseases, wherein the method comprises administering to a subject in need of such treatment a therapeutically effective amount of the optical isomers of phenylpropionic acid drugs according to claim 7.

22. A treatment method for treating one or more selected from inflammation, pain symptoms and diseases, wherein the method comprises administering to a subject in need of such treatment a therapeutically effective amount of the optical isomers of phenylpropionic acid drugs according to claim 8.
